(19) United States
(12) Patent Application Publication
(10) Pub. No.: US 2008/0262319 A1
(43) Pub. Date: Oct. 23, 2008

(54) Device for Measuring Brain Parameters

(75) Inventors: Robert Reichenberger, Wunsiedel (DE); Gerd Kunze, Zwonitz (DE); Karl-Heinz Gohler, Zwonitz (DE)

Correspondence Address: BROWDY AND NEIMARK, P.L.L.C.;624 NINTH STREET, NW, SUITE 300, WASHINGTON, DC, 20001-5303, US

(73) Assignee: Raumedic AG, Helmbrechts (DE)

(21) Appl. No.: 11/817,111

(22) PCT Filed: Jan. 21, 2006
(86) PCT No.: PCT/EP06/00527

§ 371 Date: Aug. 24, 2007

(30) Foreign Application Priority Data
Feb. 24, 2005 (DE) 10 2005 008 454.0

(51) Int. Cl. A61B 17/58; A61B 5/00

(52) U.S. Cl. 600/300; 606/301

(57) ABSTRACT

A device (1) is used for measuring brain parameters. This device has a catheter sensor unit (2) which can be implanted distally in a minimally-invasive manner in the parenchyma and/or the ventricles, guided through the cranial bone (4). The sensor unit (2) can be fixed proximally to the cranial bone. For this purpose a proximal circumferential portion of the sensor unit (2) has a self-cutting outer screw thread (5) for fastening the sensor unit (2) to the cranial bone (4). The result is a device which can be fastened in a secure, permanent and at the same time inexpensive manner preventing movement relative to the cranial bone.

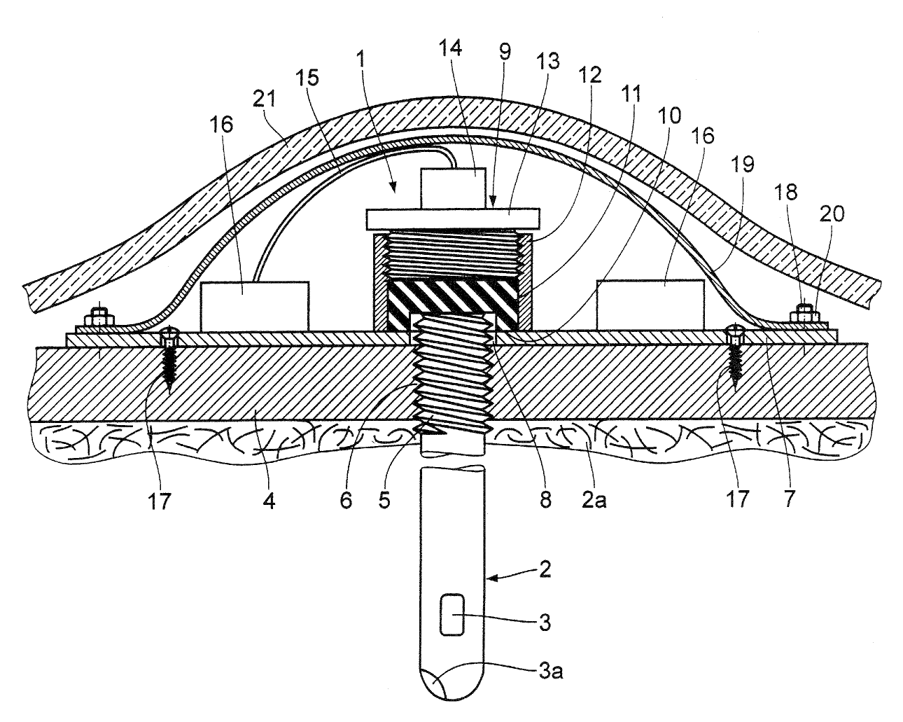

The invention relates to a device for measuring brain parameters according to the preamble of claim 1.

A device of this type is known from DE 102 39 743 A1. In brain parameter sensors of this type it is very important that the sensor unit has a secure and permanently fixed position on the cranial bone. Once implanted, any movement of the brain parameter sensor relative to the brain tissue must be avoided in order to prevent brain damage. In the known brain parameter sensors, securing the sensor unit to the cranial bone is either achieved by means of very costly securing technology or is not satisfactory with regard to prevention of relative movement.

It is therefore an object of the invention to develop a device of the type mentioned at the outset so as to ensure that it is fastened in a secure, permanent and at the same time inexpensive manner preventing relative movement.

This object is achieved according to the invention by a device with the features stated in the characterising part of claim 1.

It has been found according to the invention that the sensor unit can be fastened to the cranial bone in an inexpensive manner if the feedthrough of the sensor unit through the cranial bone is also used as a fastening point. This is achieved by using the self-cutting outer screw thread, which causes the sensor unit to be held in a permanent, secure and inexpensive manner on the cranial bone. Once implanted, undesired movement of the sensor unit relative to the brain tissue is then reliably prevented. Costly additional fastening measures can be dispensed with.

An outer screw thread according to claim 2 results in a compact device. At the same time, undesired shifting of the outer screw thread relative to the sensor unit is reliably prevented.

An outer screw thread according to claim 3 solves another important problem in the arrangement of a measuring device which can be implanted by being guided through the cranial bone, i.e. reliably sealing the sensor unit from the cranial bone, so fluid or brain tissue is reliably prevented from leaking. This sealing is achieved by arranging a sealing outer screw thread directly in the region of the feedthrough, so the requirements for a seal downstream in the proximal sensor portion are reduced or a downstream proximal seal of this type can be disposed with entirely.

An outer screw thread according to claim 4 is inexpensive to produce.

An outer screw thread according to claim 5 allows a functional separation of the various functions of said outer screw thread, i.e. cutting in on one side, and sealing on the other.

A stop according to claim 6 allows the device to be screwed in a defined manner. In the region of the stop there may also be an additional seal of the sensor unit downstream of the outer screw thread on the cranial bone in the proximal sensor portion.

An embodiment of the invention will be described in greater detail in the following with reference to the drawing.

The single FIGURE shows a cross-section through a detail of a patient's head with a device for measuring brain parameters.

The device for measuring brain parameters denoted overall by reference numeral 1 comprises a catheter sensor unit 2, which can be implanted distally in a minimally-invasive manner in the parenchyma and/or the ventricles. At its distal end, i.e. where the catheter sensor unit 2 projects into the brain tissue or fluid 2a of the patient, the catheter sensor unit 2 has a pressure sensor 3 and a temperature sensor 3a. The catheter sensor unit 2 is secured proximally to a cranial bone 4 of the patient.

In order to secure the catheter sensor unit 2 to the cranial bone 4 a proximal circumferential portion of the sensor unit 2 has a self-cutting outer screw thread 5. By cutting into the cranial bone 4, said outer screw thread produces in the cranial bone an inner screw thread 6 complementary to the outer screw thread 5.

The outer screw thread 5 is an integral component of the sensor unit 2, and is therefore not a separate component to the sensor unit. The outer screw thread 5 has a sealing coating (not shown in the drawing) so that the sensor unit 2 is sealed from the cranial bone 4 by the outer screw thread 5. It is therefore not possible for fluid or tissue to leak from the brain at the location of the implanted sensor unit 2.

The device 1 also comprises a base plate 7 having a through-hole 8 through which the catheter sensor unit 2 is guided and which is aligned with the inner screw thread 6. When the sensor unit 2 is fully screwed in, a proximal top portion 9 abuts the base plate 7 via an extension zone 10. The top portion 9 of the sensor unit 2 thus has a considerably larger circumference than the catheter portion of the sensor unit 2. The extension zone 10 is a stop for the sensor unit 2, the stop being configured at a proximal end portion, i.e. top portion 9, and delimiting the screw-in depth of said sensor unit.

In the top portion 9 an additional proximal seal 11 is arranged, which is inserted into a sleeve 12 of the top portion 9. The seal 11 also seals the sensor unit 2 from the cranial bone 4 via the base plate 7. Due to the sealing effect of the outer screw thread 5 the seal 11 can also be dispensed with. Pressure can be exerted on the seal 11 by using a screw cap 13, which is screwed into the sleeve 12 from above as shown in the drawing.

The sensor unit 2 is connected by means of a micro plug 14 and a signal line 15 to an electronics unit 16, the multiple parts of which are shown in the drawing.

The base plate 7 is fixed to the cranial bone 4 by means of screws 17. On one side, the base plate 7 has threaded bolts 18 projecting upwards as shown in the drawing. A semi-flexible cover 19, covering the device from above in the drawing, is fixed at its edge via said threaded bolts. In this way, the cover 19 is clamped at its edge between the base plate 7 and the fastening nuts 20 which are screwed onto the threaded bolts 18. The cover 19 protects the entirety of the device 1. The cover 19 is covered by the scalp 21.

When implanting the sensor unit 2 a hole is initially drilled in the cranial bone 4. The base plate 7 is subsequently fixed by means of the screws 17, such that the through-hole 8 is aligned with the hole in the cranial bone 4. The catheter portion of the sensor unit 2 is then guided through the hole in the cranial bone 4. During insertion, when the outer screw thread 5 reaches the hole in the cranial bone 4, the outer screw thread 5 is screwed in. Through the self-cutting action of the outer screw thread 5, the inner screw thread 6 is formed from the hole in the cranial bone 4. The screwing-in procedure continues until the sleeve 12 abuts the base plate 7. Lastly, after screwing on the screw cap 13, the modules of the electronics unit 16 provided on the base plate 7 are wired up. The cover 19 is subsequently fitted and the initially removed scalp is pulled back over the device 1. Said device is then ready to measure the corresponding brain pressure and brain temperature physiological parameters by means of the pressure sensor 3 and the temperature sensor 3a.

1. Device (1) for measuring brain parameters
comprising a catheter sensor unit (2), which
can be implanted distally in a minimally-invasive manner in at least one of the parenchyma and the ventricles, guided through the cranial bone (4),
can be fixed proximally to the cranial bone (4), wherein a proximal
circumferential portion of the sensor unit (2) has a self-cutting outer screw thread (5) for fixing the sensor unit (2) to the cranial bone (4).

2. Device according to claim 1, wherein the outer screw thread (5) is an integral component of the sensor unit (2).

3. Device according to claim 1, wherein the outer screw thread (5) is configured such that the sensor unit (2) can be sealed from the cranial bone (4) by means of said outer screw thread.

4. Device according to claim 3, wherein the outer screw thread (5) is made of a sealing material.

5. Device according to claim 3, wherein the outer screw thread (5) has a sealing coating.

6. Device according to claim 1, wherein, at a proximal end portion (9), the sensor unit (2) has a stop (10) which can be positioned against the cranial bone (4).
